(19) United States
(12) Patent Application Publication
(10) Pub. No.: US 2023/0277785 A1
(43) Pub. Date: Sep. 7, 2023

(54) DEVICE FOR DISPENSING A FLUID PRODUCT

(71) Applicant: APTAR FRANCE SAS, Le Neubourg (FR)

(72) Inventors: Mickaël BAILLIE, Acquigny (FR); Florent MOIA, Lyon (FR)

(73) Assignee: APTAR FRANCE SAS, Le Neubourg (FR)

(21) Appl. No.: 17/923,447

(22) PCT Filed: May 3, 2021
(86) PCT No.: PCT/FR2021/050756

§ 371 Date: Nov. 4, 2022

(30) Foreign Application Priority Data
May 5, 2020 (FR) 2004456

(51) Int. Cl. A61M 15/08; A61M 15/00; A61M 11/00

(52) U.S. Cl. CPC A61M 15/08; A61M 15/0036; A61M 11/007

(57) ABSTRACT

Device having a reservoir, a head provided with a dispensing hole, a stopper displaceable in the reservoir between a rest position and an actuation end position to dispense fluid product through the dispensing hole. The reservoir formed by a hollow, blind body having a base and a single opening which, in the rest position, is closed in a fluid-tight manner by the stopper, the device having an insert fixed in the head, the insert supporting a cannula for piercing the stopper and connecting the reservoir to the dispensing hole. The insert has at least one projection provided with a lower surface, the lower surface cooperating at the end of actuation with the reservoir in order to define the actuation end position of the stopper.

The present invention relates to a device for dispensing fluid product. More specifically, the present invention relates to a unidose or bidose device for the nasal dispensing of fluid product to dispense a pharmaceutical fluid product in the nose of a user by way of one single or two nasal spray(s).

A disadvantage of current unidose devices for the nasal dispensing of fluid product relates to the variability of the dose between different devices, in particular due to manufacturing tolerances of different parts which determine the dose end position. In addition, the dose end is generally formed by the contact between the elastomer stopper and the base of the reservoir, which involves a deformable part, which also increases the variability of the dose. The same problem is posed in a bidose, in particular relating to the second dose.

Document US2019126303 describes a prior-art device. The present invention aims to provide a fluid product dispensing device which does not reproduce the abovementioned disadvantages.

More specifically, the present invention aims to provide a fluid product dispensing device which reduces the dose variability between different devices.

The present invention also aims to provide such a device, wherein the dose end position is defined by only rigid parts of the device.

The present invention also aims to provide such a device, wherein the dose end position is independent of the actuation force exerted by the user.

The present invention also aims to provide such a device, which is simple and inexpensive to manufacture and to assemble.

The present invention therefore aims for a device for dispensing fluid product comprising a reservoir containing fluid product, a dispensing head provided with a dispensing hole, a stopper which is displaceable in said reservoir between a rest position and an actuation end position to dispense the fluid product through said dispensing hole during an actuation, said reservoir being formed by a hollow, blind body, comprising a base and a single opening which, in the rest position, is closed in a fluid-tight manner by said stopper, said device comprising a hollow insert inserted and fixed in said dispensing head, said insert supporting a cannula for piercing said stopper and thus connecting said reservoir to said dispensing hole during the actuation, said insert comprising at least one lateral projection provided with a lower surface, said lower surface cooperating at the end of actuation with said reservoir to define the actuation end position of said stopper.

Advantageously, said insert comprises a peripheral lateral projection.

In a variant, said insert comprises several separate lateral projections, distributed over the periphery of said insert.

Advantageously, said opening of said reservoir is formed by a neck provided with an upper surface, said upper surface cooperating at the end of actuation with said lower surface of said at least one lateral projection to define the actuation end position.

Advantageously, said at least one lateral projection is disposed with respect to said reservoir, such that the dose end contact is formed just before said stopper touches the base of said reservoir.

Advantageously, said reservoir contains a single fluid product dose dispensed in a single actuation.

In a variant, said reservoir contains two fluid product doses dispensed in two successive actuations.

Advantageously, said lateral projection cooperates with said reservoir after dispensing of the second dose.

These features and advantages and others of the present invention will appear more clearly during the following detailed description, made in reference to the accompanying drawings, given as non-limiting examples, and wherein:

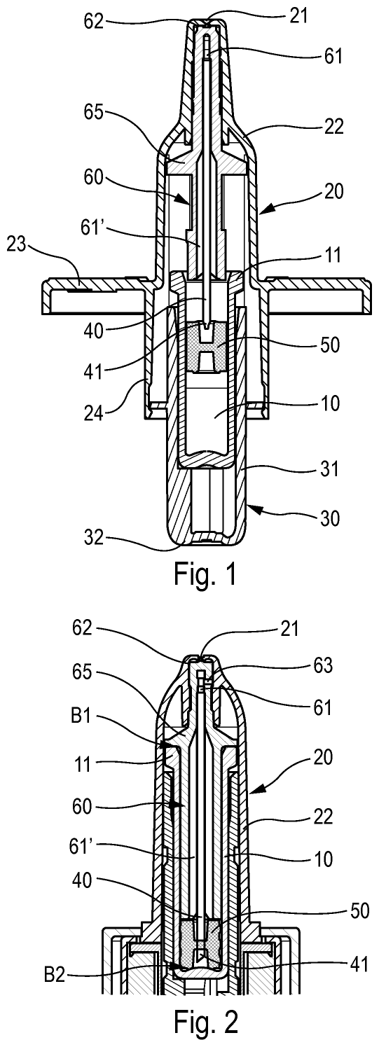

FIG. 1 is a schematic, cross-sectional view of a device for dispensing fluid product according to an advantageous embodiment, in the rest position before actuation, and

FIG. 2 is a detailed, cross-sectional view of a dispensing head and of an insert according to another advantageous embodiment, in the actuated position.

The terms “proximal” and “distal” relate to the dispensing hole. The terms “axial” and “radial” relate to the longitudinal central axis of the device. The terms “upstream” and “downstream” relate to the flow direction of the fluid during the actuation. The terms “top” and “bottom” relate to the upright position of the device represented in FIG. 1.

In reference to the figures, an advantageous embodiment of the present invention is represented, which is a unidose, i.e. that the reservoir contains a single fluid product dose dispensed in a single actuation. The present invention however also applies to bidose-type devices, wherein the reservoir contains two doses dispensed in two successive actuations.

In this embodiment, a reservoir 10 containing the fluid product to be dispensed, typically a liquid, is disposed inside a body forming a dispensing head 20. This dispensing head 20 comprises an axially oriented dispensing hole 21. This dispensing hole 21 serves to dispense a fluid product dose outside of said dispensing head 20 during the actuation of the device by a user.

The dispensing head 20 comprises an extended nosepiece 22 comprising at its proximal axial end, said dispensing hole 21, as well as a lateral body 24, connected to said nosepiece 22 at a radial flange 23.

A hollow insert 60 is disposed in the dispensing head 20, upstream from said dispensing hole 21, said hollow insert 60 defining an expulsion channel 61, 61′ and, cooperating with the base wall of the dispensing head 20, a spray profile 62 directly upstream from said dispensing hole 21. At least one radial opening 63, disposed in the proximity of the proximal end of the insert 60, allows the fluid to pass from the expulsion channel 61 to the spray profile 62. The expulsion channel comprises a proximal channel part 61, at said radial opening 63. The expulsion channel comprises a distal channel part 61′ of diameter greater than that of the proximal channel part 61.

According to the invention, on its external wall, the insert 60 comprises a lateral projection 65, extending preferably over the whole periphery. This lateral projection 65 is made of a one-piece part with the insert 60 and is not detachable or removable from said insert, as it would be, for example, for a divisible bridge. The lateral projection 65 comprises an upper surface and a lower surface.

A hollow cannula 40 is inserted and fixed in the proximal part 61 of said expulsion channel, said cannula 40 comprising at its distal end, a piercing tip 41. Advantageously, the cannula 40 is forcefully fitted in the proximal channel part 61. Thus, the insertion of the cannula 40 in the insert 60 is facilitated, the forceful fitting being limited to the proximal channel part 61.

The reservoir 10 is formed by a hollow, blind body, comprising a single opening, closed by a plunger-stopper 50. In the example represented, which is a unidose, the reservoir 10 contains a single fluid product dose to dispense with one single actuation of the device. In the case of a bidose, the reservoir contains two doses, and the device comprises dose splitting means to ensure the dispensing of a single dose at each actuation. The opening of the reservoir is formed by a neck 11 provided with an upper surface. The plunger-stopper 50 can be displaced in the reservoir 10 between a rest position and an actuation end position to dispense said fluid product through the dispensing hole 21 during the actuation.

During the actuation, the reservoir 10 is axially displaced upwards with respect to the dispensing head 20 and therefore with respect to the cannula 40. The plunger-stopper 50 is thus pushed against the tip 41 of the cannula 40 to be pierced, which allows the expulsion of all or some of the fluid product contained in the reservoir 10.

Actuation means 30 are provided to allow the actuation of the device.

In the embodiment of FIG. 1, these actuation means 30 comprise an actuation body 31 which is movable with respect to the dispensing head 20, said actuation body 31 cooperating with said reservoir 10 to axially displace it with respect to the dispensing head 20, in the direction of the dispensing hole 21.

The operation of the unidose device represented in the figures will be detailed below.

Conventionally, the user places two fingers on the radial flange 23 formed on the dispensing head 20, and presses with their thumb on the distal axial bottom 32 of said actuation body 31. During such an actuation, the reservoir 10 is therefore axially pushed in the direction of the dispensing hole 21, such that the cannula 40 pierces the plunger-stopper 50. The content of the reservoir 10 is thus connected to the dispensing hole 21 and the press of the user on the actuation body 31 displaces the plunger-stopper 50 in the reservoir 10 to ensure the dispensing of the fluid product.

The actuation end position is defined by the mechanical abutment between the upper surface of the neck 11 of the reservoir 10 and the lower surface of the lateral projection 65 of the insert 60.

Thus, as can be seen in FIG. 2, the actuation end position is defined by only two rigid parts of the device which form an abutment B1 blocking the axial displacement of the reservoir 10 during the actuation. This actuation end position is consequently independent from the actuation force exerted by the user to displace the reservoir 10.

The lateral projection 65 is disposed with respect to the reservoir 10, such that the dose end contact B1 is formed just before the stopper 50 touches the base of the reservoir 10.

Thus, at B2 in FIG. 2, there is no contact and therefore no compression or deformation of the stopper 50 at the dose end, which can generate a dose variability.

In the examples represented in the figures, the abutment surfaces of the reservoir 10 and of the lateral projection 65 are substantially horizontal in the upright position represented in the figures. In a variant, inclined surfaces could be imagined complementarily. Also, a plurality of non-peripheral lateral projections 65, for example two, three or four projections distributed over the periphery of the insert 60 could be imagined, and cooperating in the dose end position with the upper surface of the neck 11 of the reservoir 10.

The present invention therefore provides a device for which the actuation end position is defined only by two rigid parts which abut against one another at the actuation stroke end. This makes the device more robust and reliable, and guarantees reproducible performances. The dose variability is thus reduced by at least 25%, advantageously by at least 50%, preferably around 60% with respect to standard unidose or bidose devices in which the dose end is defined by the contact between the elastomer stopper and the base of the reservoir.

Naturally, other embodiment variants can also be considered, without moving away from the scope of the present invention, such as defined by the accompanying claims.

1. A device for dispensing fluid product comprising a reservoir containing the fluid product, a dispensing head provided with a dispensing hole, a stopper which is displaceable in said reservoir between a rest position and an actuation end position to dispense the fluid product through said dispensing hole during an actuation, said reservoir being formed by a hollow, blind body, comprising a base and a single opening which, in the rest position, is closed in a fluid-tight manner by said stopper, said device comprising a hollow insert inserted and fixed in said dispensing head, said insert supporting a cannula for piercing said stopper and thus connecting said reservoir to said dispensing hole during the actuation, characterised in that said insert comprises at least one lateral projection provided with a lower surface, said lower surface cooperating at the end of actuation with said reservoir to define the actuation end position of said stopper.

2. The device according to claim 1, wherein said insert comprises a peripheral lateral projection.

3. The device according to claim 1, wherein said insert comprises several separate lateral projections, distributed over the periphery of said insert.

4. The device according to claim 1, wherein said opening of said reservoir is formed by a neck provided with an upper surface, said upper surface cooperating at the end of actuation with said lower surface of said at least one lateral projection to define the actuation end position.

5. The device according to claim 1, wherein said at least one lateral projection is disposed with respect to said reservoir, such that the dose end contact is formed just before said stopper touches the base of said reservoir.

6. The device according to claim 1, wherein said reservoir contains a single fluid product dose dispensed in a single actuation.

7. The device according to claim 1, wherein said reservoir contains two fluid product doses dispensed in two successive actuations.

8. The device according to claim 7, wherein said lateral projection cooperates with said reservoir after dispensing of the second dose.
